(19) United States
(12) Patent Application Publication
(10) Pub. No.: US 2025/0255465 A1
(43) Pub. Date: Aug. 14, 2025

(54) ENDOSCOPY IMAGING SYSTEM INCLUDING IN-LINE DUAL CAMERA 3D ENDOSCOPY CANNULA ASSEMBLY

(71) Applicant: Raytrx, LLC, Tulsa, OK (US)

(72) Inventors: David Kessler, Rochester, NY (US); Michael A. H. Freeman, Tulsa, OK (US); Mitchael C. Freeman, Tulsa, OK (US); Jordan Boss, Tulsa, OK (US)

(21) Appl. No.: 19/048,746

(22) Filed: Feb. 7, 2025

Related U.S. Application Data
(60) Provisional application No. 63/551,928, filed on Feb. 9, 2024.

(51) Int. Cl. A61B 1/00; A61B 1/05; A61B 17/34

(52) U.S. Cl. CPC A61B 1/00087; A61B 1/00096; A61B 1/00194; A61B 1/05; A61B 17/3421

(57) ABSTRACT

An endoscopy cannula assembly is described herein. The endoscopy cannula assembly includes a cannula extending along a longitudinal axis and a 3D imaging system positioned within the cannula. The 3D imaging system includes a pair of imaging modules orientated in an opposing mirrored relationship along the longitudinal axis. Each imaging module includes a camera sensor orientated along a first optical axis and an optical engine configured to receive light rays through an opening defined along a sidewall of the cannula along a second optical axis and direct the received light rays along the first optical axis towards the camera sensor.

### CROSS REFERENCE TO RELATED APPLICATIONS

This application claims the benefit of U.S. Provisional application Ser. No. 63/551,928, filed on Feb. 9, 2024, the disclosures of which are hereby incorporated by reference in their entirety and for all purposes.

### COPYRIGHT NOTICE

A portion of this disclosure contains material that is subject to copyright protection. The copyright owner has no objection to the facsimile reproduction by anyone of this patent document as it appears in the U.S. Patent and Trademark Office, patent file, or records, but reserves all copyrights whatsoever in the subject matter presented herein.

### TECHNICAL FIELD

The present disclosure generally relates to cannulas for use in endoscopes and, more specifically, to endoscopy imaging systems including endoscopy cannula assemblies with in-line dual camera 3D imaging for use in ocular endoscopy.

### BACKGROUND OF THE INVENTION

There are three major categories in endoscopy in general: 1) Fiber based-camera at the proximal end and fibers are used for imaging and illumination with some having these features: a) Low gauge (such as in laparoscopic endoscopy), b) Flexible, c) Illumination provided inside the cannula, d) 3D available in some as side by side double apertures, e) Zoom is provided by some, and f) Variable focusing may be available; 2) Rigid Hopkins relay based-camera at the proximal end with some having these features: a) Bendable tips available, b) Zooming, c) Variable focusing, and d) Large field-of-view (FOV); and 3) Digital camera at the distal tip, Mono only (no 3D).

Currently the preferred gauges in Ocular Endoscopy are 23 (0.6 mm) 25 (0.5 mm) and 27 (0.45 mm) which do not necessitate stitches upon removal. At these sizes, 3D based on two lenses side by side is not possible since such an arrangement does not provide enough base distance for good stereopsis. A common convergence angle is 60 mm IPD over 10 inches which is about 14°. If the intraocular working distance is say 10 mm, the “IPD” at the distal tip would be 2.4 mm to provide 14° convergence.

In addition, at least some known ophthalmic optical coherence tomography (OCT) scans use small diameter which do provide depth info but which have issues like color, lag, registration, and FOV.

The present invention solves one or more of the problems identified above.

### SUMMARY OF INVENTION

In one aspect of the present invention, an endoscopy cannula assembly is provided. The endoscopy cannula assembly includes a cannula extending along a longitudinal axis and a 3Dimensional (3D) imaging system positioned within the cannula. The 3D imaging system includes a pair of imaging modules orientated in an opposing mirrored relationship along the longitudinal axis. Each imaging module includes a camera sensor orientated along a first optical axis and an optical engine configured to receive light rays through an opening defined along a sidewall of the cannula along a second optical axis and direct the received light rays along the first optical axis towards the camera sensor.

In another aspect of the present invention, an endoscopic imaging apparatus is provided. The endoscopic imaging apparatus includes a handle subsystem including a control handle configured for attachment and actuation of endoscopic tools, a shaft extending outwardly from the handle subsystem between a proximal end opposite a distal end, and an endoscopic tool. The endoscopic tool includes a trocar piercing tip coupled to the distal end of the shaft and movable between an open position and a closed position and an endoscopy cannula assembly positioned within the shaft and extendable through the trocar piercing tip. The endoscopy cannula assembly includes a cannula extending along a longitudinal axis and a 3D imaging system positioned within the cannula. The 3D imaging system includes a pair of imaging modules orientated in an opposing mirrored relationship along the longitudinal axis. Each imaging module includes a camera sensor orientated along a first optical axis and an optical engine configured to receive light rays through an opening defined along a sidewall of the cannula along a second optical axis and direct the received light rays along the first optical axis towards the camera sensor.

### BRIEF DESCRIPTION OF THE DRAWINGS

Non-limiting and non-exhaustive embodiments of the present invention are described with reference to the following figures. Other advantages of the present disclosure will be readily appreciated, as the same becomes better understood by reference to the following detailed description when considered in connection with the accompanying drawings wherein:

FIG. 1 is a schematic view of an endoscopy cannula assembly, according to embodiments of the present invention;

FIGS. 2-7 are partial perspective views of the endoscopy cannula assembly shown in FIG. 1 including a 3D imaging system including a pair of imaging modules, according to embodiments of the present invention;

FIG. 8 is an exploded view of an imaging module shown in FIGS. 2-7;

FIGS. 9-12 are perspective views of an endoscopic imaging apparatus including the endoscopy cannula assembly, according to embodiments of the present invention; and

FIGS. 13-14 are schematic views of a visualization theater that may be used with the endoscopic imaging apparatus show in FIGS. 8-12, according to embodiments of the present invention.

Corresponding reference characters indicate corresponding parts throughout the drawings.

### DETAILED DESCRIPTION

With reference to the figures, and in operation, the present invention is directed towards an endoscopic imaging apparatus including an endoscopy cannula assembly that includes an in-line dual camera 3D imaging system for use in ocular endoscopy.

Currently, known ocular endoscopy using high gauge/small diameter cannulas are mono-scopes since the common 3D capture is done by two side by side cameras which is impossible with small diameter cannulas. The endoscopy cannula assembly of the present invention provides the combination of a small diameter cannula and 3D capture with two in-line cameras. The stereoscopic in-line arrangement provides 3D capture with two in line cameras and the small diameter cannulas (e.g., 0.8 mm).

For example, the cannula may be Gauge 23 and may be disposable, include a disposable cover, or be autoclavable. The 3D imaging system may include 3D with adjustable IPD for 2 to 4 mm, dual in line cameras rotatable about z-axis. The 3D imaging system may also include 3D on a bendable distal head, All digital with >10,000 pixels, and variable focus (e.g., piezos). The 3D imaging system may also include a FOV with 32 degrees on the diagonal, 22.5×22.5 degrees, with 5 mm for the working distance, and a convergence angle of 14 degrees which will provide good stereopsis. For example, the 3D imaging system may be designed for 5 mm working distance from the retina during ocular endoscopy. This design is for a field of view of 22.5×22.5 degrees inside the eyeball.

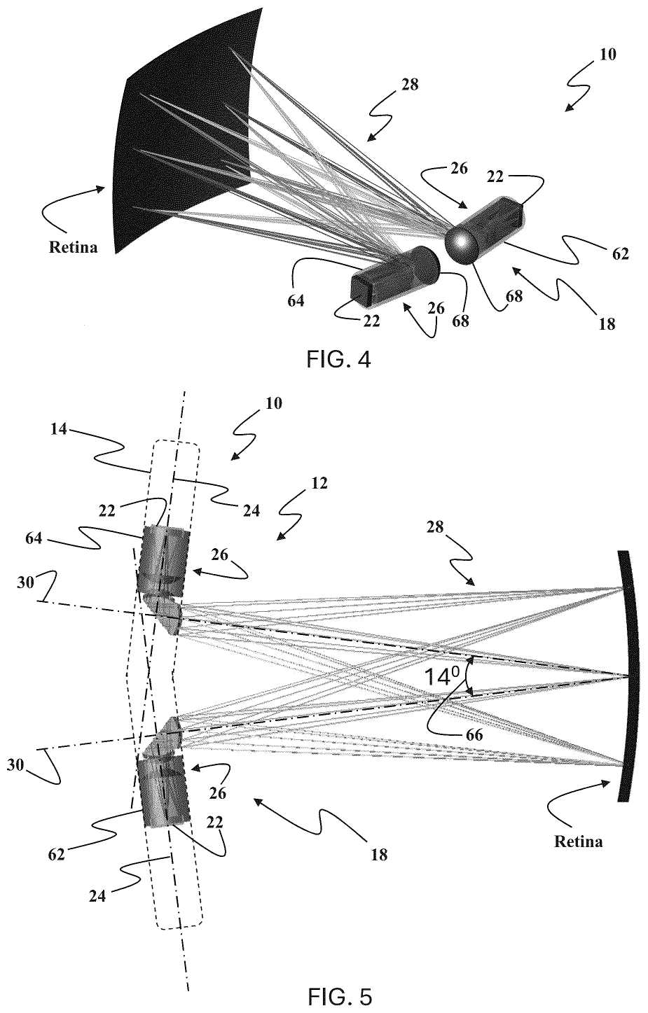

In some embodiments, the 3D imaging system 18 may include two modules that are co-linear (shown in FIG. 2), so the field of view is partially 3D flanked by two 2D domains. In other embodiments, the 3D imaging system may include two modules that are angled so the full retina field is in 3D (shown in FIGS. 4 and 5). In addition, the optics apertures may be 0.35×0.35 mm and the camera sensors may be 0.35×0.35 mm, and are inside the inner diameter of the cannula of 0.5 mm with an external diameter of 0.6 mm. Between the optics and the cannula there is room for wires.

The following is a detailed description of the preferred embodiments of the disclosure, reference being made to the figures in which the same reference numerals identify the same elements of structure in each of the several figures.

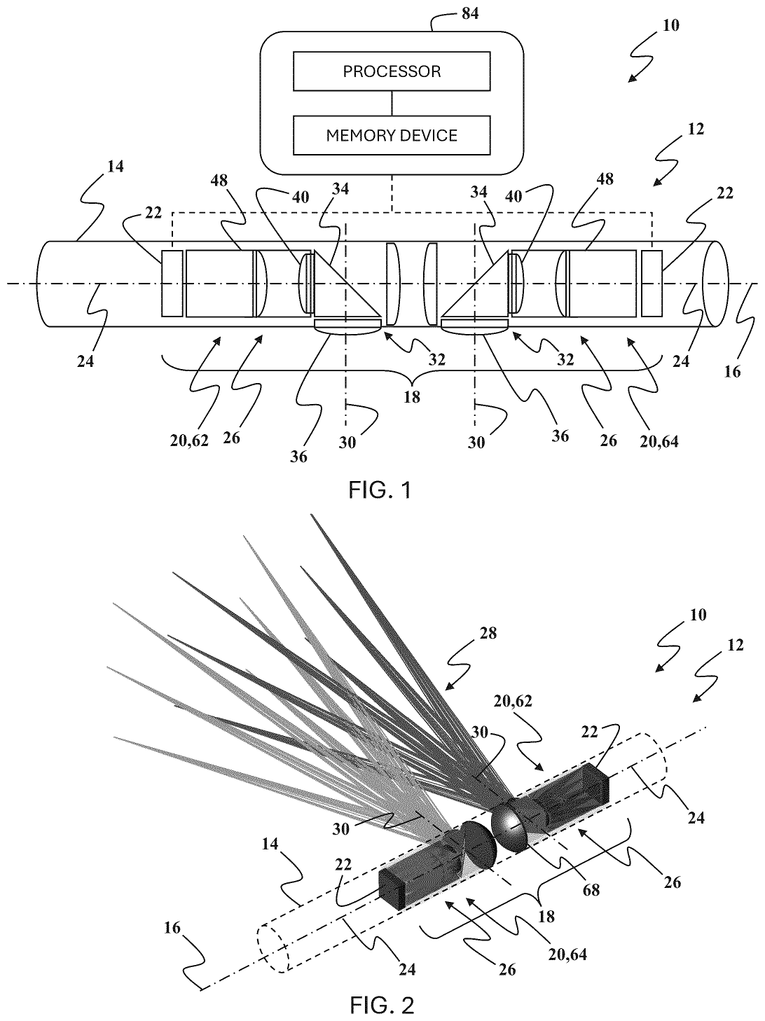

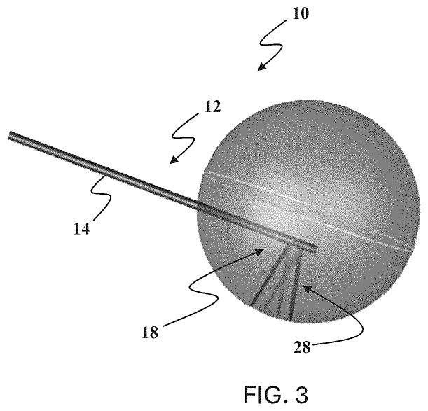

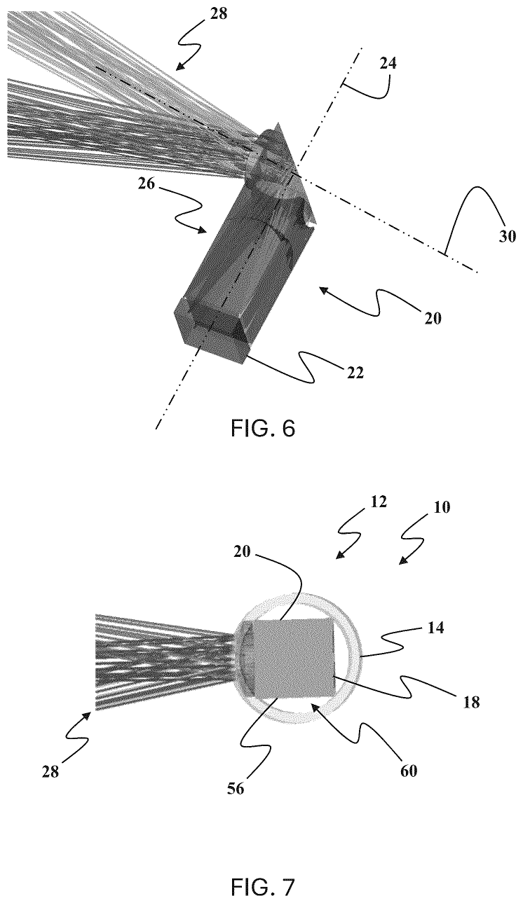

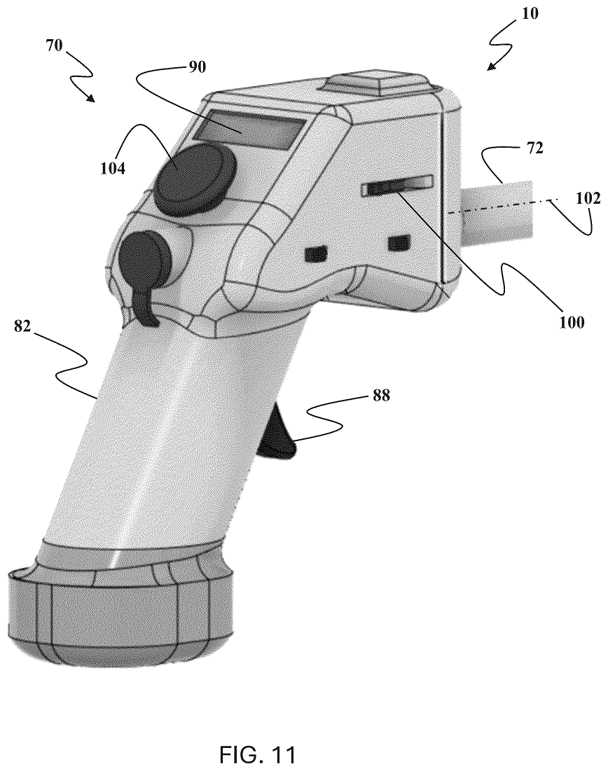

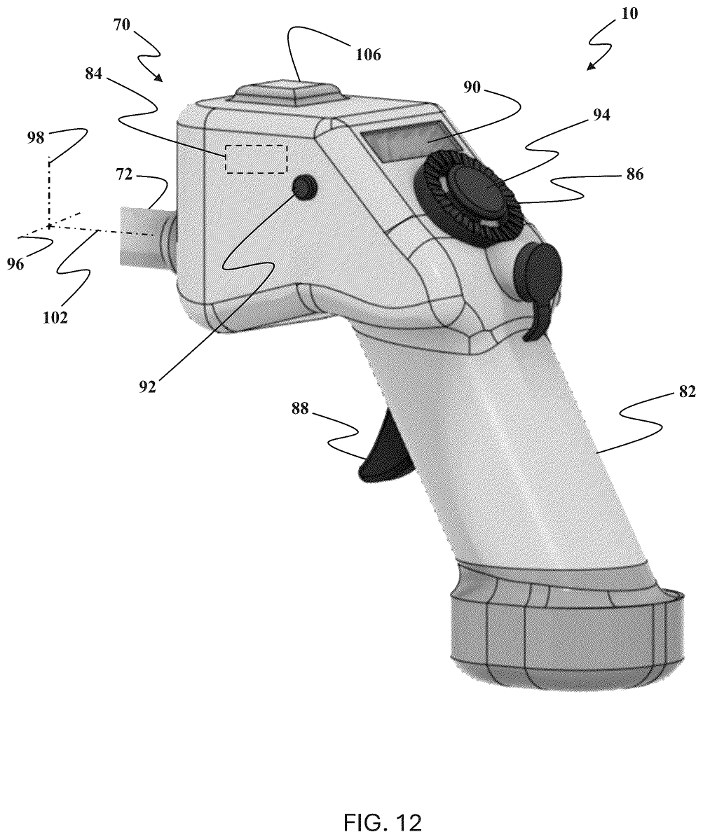

Referring to FIGS. 1-14, in the illustrated embodiment, the endoscopic imaging apparatus 10 includes an endoscopy cannula assembly 12 including a cannula 14 extending along a longitudinal axis 16 and an in-line dual camera 3D imaging system 18 positioned within the cannula 14. The 3D imaging system 18 includes a pair of imaging modules 20 orientated in an opposing mirrored relationship along the longitudinal axis 16.

In the illustrated embodiment, each imaging module 20 includes a camera sensor 22 orientated along a first optical axis 24 and an optical engine 26 configured to receive light rays 28 along a second optical axis 30 and direct the received light rays along the first optical axis 24 towards the camera sensor 22. For example, the cannula 14 may include one or more openings 32 defined along a sidewall of the cannula 14. The optical engine 26 receives the light rays 28 through the one or more openings 32 defined along the cannula 14 along the second optical axis 30 and directs the received light rays along the first optical axis 24 towards the camera sensor 22.

In the illustrated embodiment, the optical engine 26 includes a folding prism 34 spaced a distance from the camera sensor 22 along the first optical axis 24. The folding prism 34 is configured to direct the light rays received along the second optical axis 30 towards the camera sensor 22. In this way, the second optical axis 30 may be orientated perpendicular to the first optical axis 24 such the 3D imaging system 18 may include dual in-line opposing camera sensors 22 positioned within the cannula 14.

The optical engine 26 also includes a first plano aspheric lens 36 positioned adjacent to an entrance face 38 of the folding prism 34 and a second plano aspheric lens 40 positioned adjacent an exit face 42 of the folding prism 34. The entrance face 38 of the folding prism 34 is defined along the second optical axis 30. The first plano aspheric lens 36 is orientated along the second optical axis 30 such that a flat surface 44 of the first plano aspheric lens 36 is adjacent to the entrance face 38 of the folding prism 34. In some embodiments, the flat surface 44 of the first plano aspheric lens 36 is cemented to the entrance face 38 of the folding prism 34.

The exit face 42 of the folding prism 34 is defined along the first optical axis 24. The second plano aspheric lens 40 is orientated along the first optical axis 24 such that a flat surface 46 of the second plano aspheric lens 40 is adjacent to the exit face 42 of the folding prism 34. In some embodiments, the flat surface 46 of the second plano aspheric lens 40 is cemented to the exit face 42 of the folding prism 34.

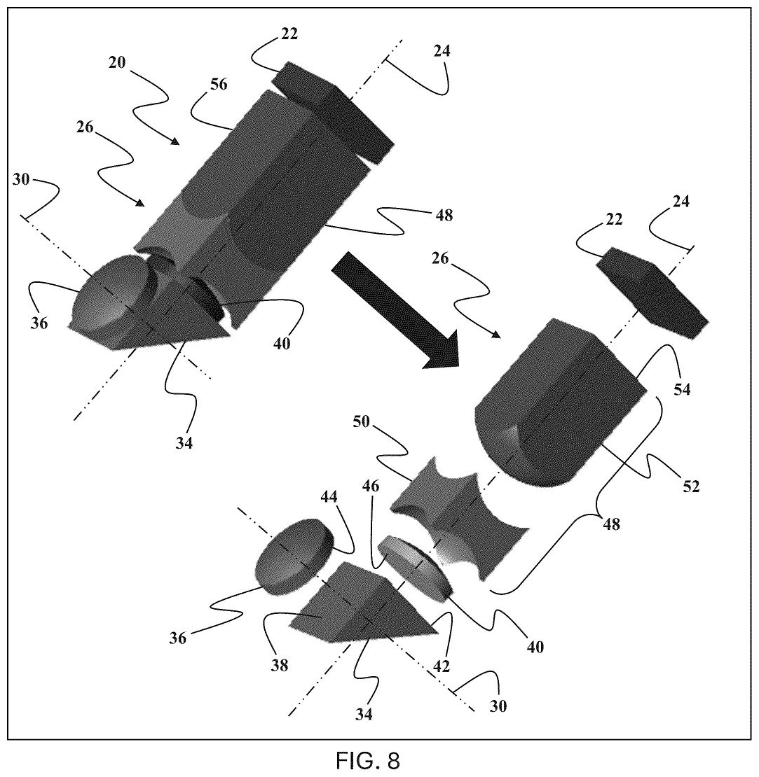

The optical engine 26 also includes a doublet 48 orientated along the first optical axis 24 and positioned between the second plano aspheric lens 40 and the camera sensor 22. The doublet 48 includes a bi-concave aspheric lens 50 and a plano convex lens 52 positioned between the bi-concave aspheric lens 50 and the camera sensor 22, with a flat surface 54 of the plano convex lens 52 orientated towards the camera sensor 22. In some embodiments, the bi-concave aspheric lens 50 is cemented to the plano convex lens 52 to form the doublet 48. For example, as shown in FIG. 8, the two plano aspheric lenses 36, 40 are cemented to the folding prism 34, and the bi-concave aspheric lens 50 and the plano convex lens 52 are cemented to form a doublet 48.

In the illustrated embodiment, the doublet 48 includes a substantially rectangular body 56 extending along the first optical axis 24 such that a gap 60 is defined between the inner surface of the cannula 14 and the doublet body 56. The gap 60 is sized and shaped to accommodate communication and/or power wires connected to the camera sensor 22. For example, the optics apertures may be 0.35×0.35 mm and are inside the inner diameter of the cannula of 0.5 mm, with an external diameter is 0.6 mm, forming the gap 60 between the optics and the cannula for room for wires.

In some embodiments, the pair of imaging modules 20 includes a first imaging module 62 that is axially aligned with a second imaging module 64. For example, as shown in FIG. 2, the 3D imaging system 18 may include the first imaging module 62 axially aligned with the second imaging module 64 such that the first optical axis 24 of each imaging module 62, 64 is co-axial, and the second optical axis 30 of each imaging module 62, 64 is substantially parallel to each other.

In other embodiments, for example, as shown in FIGS. 4-5, the first imaging module 62 may be obliquely aligned with the second imaging module 64 such that the first optical axis 24 of each imaging module 62, 64 are obliquely orientated to each other, and the second optical axis 30 of each imaging module 62, 64 intersect to form a convergence angle 66. For example, as shown in FIG. 5, the convergence angle 66 formed by the two intersecting second optical axes 30 defines a convergence angle 66 equal to about 14 degrees, which is also the angle a normal 65 mm human interpupillary distance (IPD) will suspend when looking at an object at 10″. In some embodiments, the endoscopy cannula assembly 12 may include a piezo motor and/or gimble assembly coupled to the pair of imaging modules 20 to change a position of the pair of imaging modules 20 over couple of mm of focusing with a slight change in sensor axial position.

In some embodiments, each imaging module 20 may include an end cap 68 positioned adjacent to the folding prism 34 to facilitate preventing light rays from entering the opposing imaging module 20.

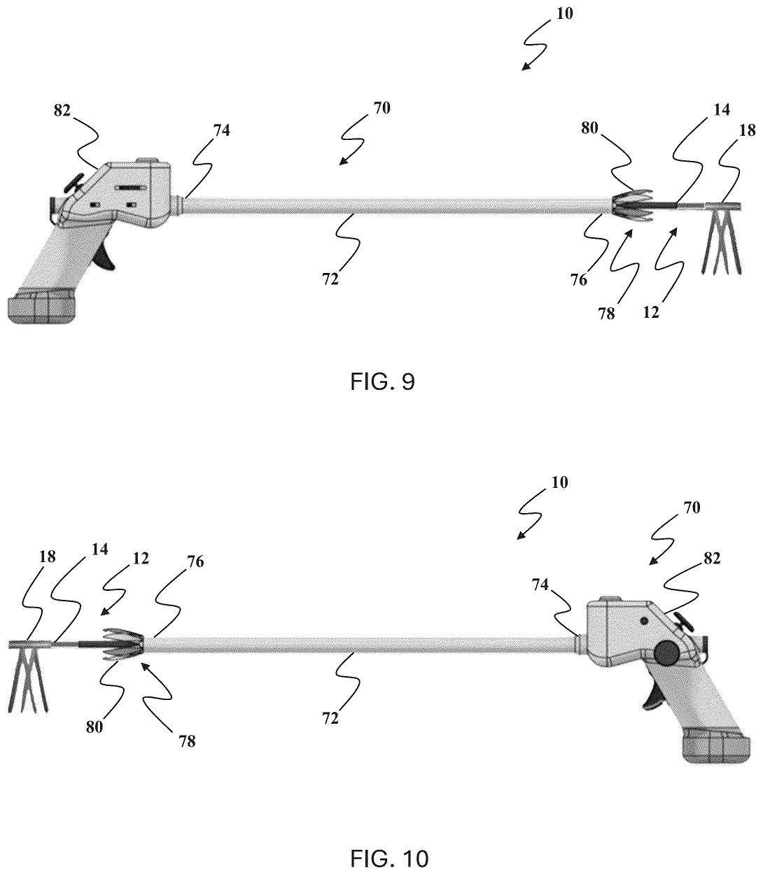

Referring to FIGS. 9-12, in the illustrated embodiment the endoscopic imaging apparatus 10 also includes a handle subsystem 70, a shaft 72 extending outwardly from the handle subsystem 70 between a proximal end 74 and an opposite a distal end 76, and an endoscopic tool 78. The endoscopic tool 78 includes a trocar piercing tip 80 coupled to the distal end 76 of the shaft 72 and movable between an open position and a closed position, and the endoscopy cannula assembly 12 positioned within the shaft 72 and extendable through the trocar piercing tip 80.

The handle subsystem 70 includes a control handle 82 that is configured for attachment and actuation of endoscopic tool 78. The control handle 82 includes a controller 84 coupled to the camera sensors 22, and including a memory device coupled to one or more processors that are programmed to receive image data from the camera sensors 22 and store the received image data in the memory device.

The handle subsystem 70 includes with one or more batteries, controllers, circuit boards and wireless modules, camera modules (sensor, camera, and circuit/power board and lens), and antennas for sending and receiving data and video. The control handle 82 may also house and control all the subsystems related to the endoscopic tool 78 manipulation. For example, the control handle 82 may include a control roll wheel 86 disposed on the side of control handle 82 and a safety trigger 88. The roll wheel 86 can be used to roll the endoscopic tool 78 360 degrees about the longitudinal axis 16. The safety trigger 88 provides a mechanical interlock for roll wheel 86 rotation, locked in position unless safety trigger 88 is actuated.

The control handle 82 may also include a digital display 90 providing a readout panel for display of information on instrument configuration and positioning. The digital display 90 may indicate tool extension distance, such as in Imperial or SI Units. For example, when in piercing tool mode, the digital display 90 can show the percentage for tool opening. Units of measurement, such as Imperial or SI Units, can be selected using a units button 92 as a type of control toggle.

The control handle 82 may also include a joystick control 94 that can be used to control movement of the endoscopic tool 78 about an x-axis 96 and/or a y-axis 98. The joystick control 94 can be enabled by actuating safety trigger 88. The control handle 82 may also include an extension wheel 100 that can be used to move the endoscopy cannula assembly 12 forward or backward along a z-axis 102 defined along a direction of the shaft 72.

Additional details of the handle subsystem 70, which may be used in the present invention, are described in U.S. patent application Ser. No. 17/473,689 to David Street et al., filed Sep. 13, 2021, titled “Wireless Laparoscopic Device with Gimballed Camera”, which is incorporated herein by reference in its entirety.

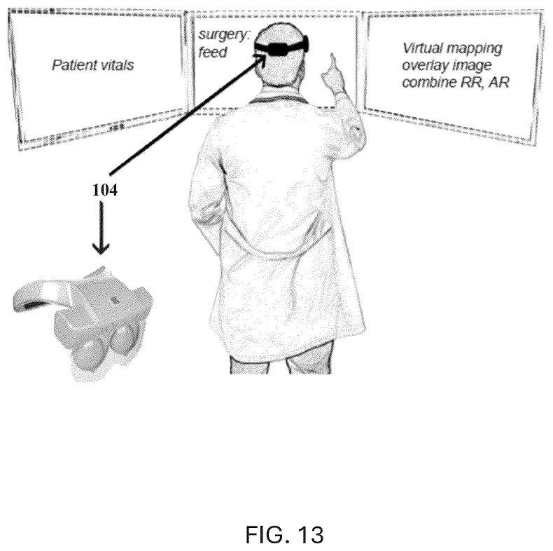

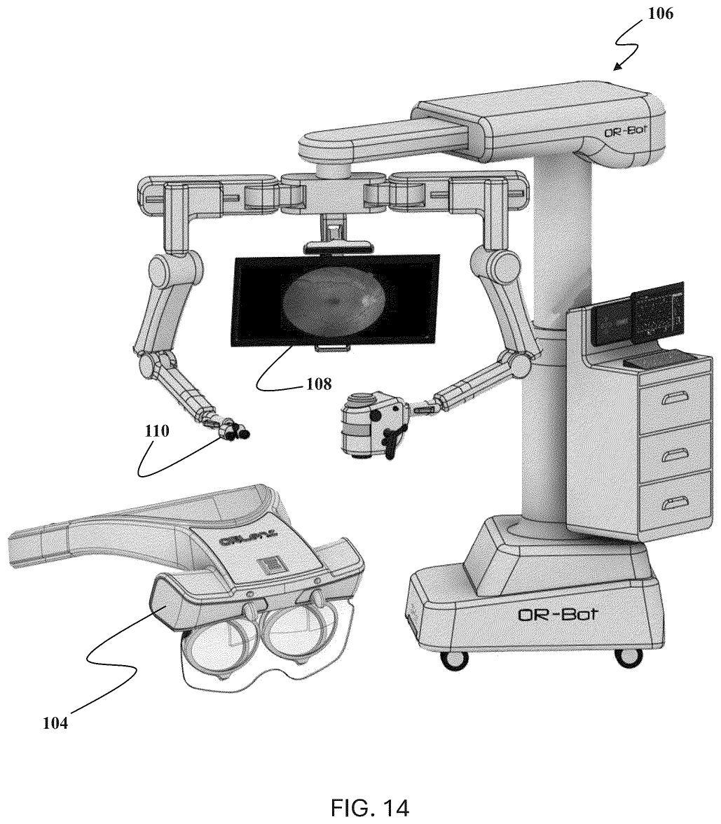

Referring to FIGS. 13-14, in some embodiments, the handle subsystem controller 84 may be coupled in communication with a wearable augmented/extended reality (AXR) headset 104 adapted to be worn by a user. For example, the control handle 82 may include a wireless transceiver 106 coupled to the controller 84 for use in wirelessly transmitting the image data received from the camera sensors 22 to the AXR headset 104 for viewing by the user. Additional details of the AXR headset 104, which may be used in the present invention, are described in U.S. patent application Ser. No. 17/139,167 to David Kessler et al., filed Dec. 31, 2020, titled “Wearable Pupil-Forming Apparatus”; and U.S. patent application Ser. No. 18/531,248 to David Kessler et al., filed Dec. 6, 2023, titled “Augmented Reality Near-Eye Pupil-Forming Catadioptric Optical Engine in Glasses Format”, which are incorporated herein by reference in their entirety. The AXR headset 104 may be, for example, the ORLenz™ Extended Reality Visualization headset sold by Ocutrx™.

In some embodiments, the controller 84 may be coupled in communication with a controller of an All-Digital Multi-Option 3D Viewing Theatre (ADMO3DV) system 106 including a 3D autostereoscopic monitor (3DAM) 108, a 3D digital viewport (3DDV) device 110, and the AXR headset 104. The controller 84 may be programmed to transmit the image data received from the camera sensors 22 to the controller of the ADMO3DV system 106 to display the received images on the 3D autostereoscopic monitor 108, the 3D digital viewport device 110, and/or the AXR headset 104.

Additional details of the All-Digital Multi-Option 3D Viewing Theatre system 106, which may be used in the present invention, are described in U.S. patent application Ser. No. 18/450,997 to Michael H. Freeman et al., filed Sep. 6, 2023, titled “Surgery Visualization Theatre”, which is incorporated herein by reference in its entirety. The All-Digital Multi-Option 3D Viewing Theatre system 106 may be, for example, the OR-Bot™ sold by Ocutrx™.

A controller, computing device, or computer, such as described herein, includes at least one or more processors or processing units and a system memory. The controller typically also includes at least some form of computer readable media. By way of example and not limitation, computer readable media may include computer storage media and communication media. Computer storage media may include volatile and nonvolatile, removable and non-removable media implemented in any method or technology that enables storage of information, such as computer readable instructions, data structures, program modules, or other data. Communication media typically embody computer readable instructions, data structures, program modules, or other data in a modulated data signal such as a carrier wave or other transport mechanism and include any information delivery media. Those skilled in the art should be familiar with the modulated data signal, which has one or more of its characteristics set or changed in such a manner as to encode information in the signal. Combinations of any of the above are also included within the scope of computer readable media.

The order of execution or performance of the operations in the embodiments of the invention illustrated and described herein is not essential, unless otherwise specified. That is, the operations described herein may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

In some embodiments, a processor, as described herein, includes any programmable system including systems and microcontrollers, reduced instruction set circuits (RISC), application specific integrated circuits (ASIC), programmable logic circuits (PLC), and any other circuit or processor capable of executing the functions described herein. The above examples are exemplary only, and thus are not intended to limit in any way the definition and/or meaning of the term processor.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Other aspects and features of the present invention can be obtained from a study of the drawings, the disclosure, and the appended claims. The invention may be practiced otherwise than as specifically described within the scope of the appended claims. It should also be noted that the steps and/or functions listed within the appended claims, notwithstanding the order of which steps and/or functions are listed therein, are not limited to any specific order of operation.

Although specific features of various embodiments of the invention may be shown in some drawings and not in others, this is for convenience only. In accordance with the principles of the invention, any feature of a drawing may be referenced and/or claimed in combination with any feature of any other drawing.

The invention has been described in detail with particular reference to a presently preferred embodiment, but it will be understood that variations and modifications can be effected within the spirit and scope of the disclosure. The presently disclosed embodiments are therefore considered in all respects to be illustrative and not restrictive. The scope of the invention is indicated by any appended claims, and all changes that come within the meaning and range of equivalents thereof are intended to be embraced therein.

What is claimed is:

1. An endoscopy cannula assembly, comprising:
a cannula extending along a longitudinal axis; and
a 3-dimensional imaging system positioned within the cannula and including:
a pair of imaging modules orientated in an opposing mirrored relationship along the longitudinal axis, each imaging module including:
a camera sensor orientated along a first optical axis; and
an optical engine configured to receive light rays through an opening defined along a sidewall of the cannula along a second optical axis and direct the received light rays along the first optical axis towards the camera sensor.

2. The endoscopy cannula assembly of claim 1, wherein the optical engine includes:
a folding prism spaced a distance from the camera sensor along the first optical axis and configured to direct the light rays received along the second optical axis towards the camera sensor.

3. The endoscopy cannula assembly of claim 2, wherein the optical engine includes:
a first plano aspheric lens adjacent an entrance face of the folding prism defined along the second optical axis; and
a second plano aspheric lens adjacent an exit face of the folding prism defined along the first optical axis.

4. The endoscopy cannula assembly of claim 3, wherein the optical engine includes:
a flat surface of the first plano aspheric lens cemented to the entrance face of the folding prism; and
a flat surface of the second plano aspheric lens cemented to the exit face of the folding prism.

5. The endoscopy cannula assembly of claim 3, wherein the optical engine includes:
a doublet positioned between the second plano aspheric lens and the camera sensor.

6. The endoscopy cannula assembly of claim 5, wherein the optical engine includes:
the doublet including a bi-concave aspheric lens cemented to a plano convex lens positioned between the bi-concave aspheric lens and the camera sensor.

7. The endoscopy cannula assembly of claim 6, wherein a flat surface of the plano convex lens is orientated towards the camera sensor.

8. The endoscopy cannula assembly of claim 5, wherein the doublet includes a substantially rectangular body extending along the first optical axis.

9. The endoscopy cannula assembly of claim 1, wherein the pair of imaging modules includes a first imaging module axially aligned with a second imaging module.

10. The endoscopy cannula assembly of claim 1, wherein the pair of imaging modules includes a first imaging module obliquely aligned with a second imaging module.

11. An endoscopic imaging apparatus comprising:
a handle subsystem including a control handle configured for attachment and actuation of endoscopic tools;
a shaft extending outwardly from the handle subsystem between a proximal end opposite a distal end; and
an endoscopic tool including:
a trocar piercing tip coupled to the distal end of the shaft and movable between an open position and a closed position; and
an endoscopy cannula assembly positioned within the shaft and extendable through the trocar piercing tip, the endoscopy cannula assembly including:
a cannula extending along a longitudinal axis; and
a 3-dimensional imaging system positioned within the cannula and including:
a pair of imaging modules orientated in an opposing mirrored relationship along the longitudinal axis, each imaging module including:
a camera sensor orientated along a first optical axis; and
an optical engine configured to receive light rays through an opening defined along a sidewall of the cannula along a second optical axis and direct the received light rays along the first optical axis towards the camera sensor.

12. The endoscopic imaging apparatus of claim 11, wherein the optical engine includes:
a folding prism spaced a distance from the camera sensor along the first optical axis and configured to direct the light rays received along the second optical axis towards the camera sensor.

13. The endoscopic imaging apparatus of claim 12, wherein the optical engine includes:
a first plano aspheric lens adjacent an entrance face of the folding prism defined along the second optical axis; and
a second plano aspheric lens adjacent an exit face of the folding prism defined along the first optical axis.

14. The endoscopic imaging apparatus of claim 13, wherein the optical engine includes:
a flat surface of the first plano aspheric lens cemented to the entrance face of the folding prism; and
a flat surface of the second plano aspheric lens cemented to the exit face of the folding prism.

15. The endoscopic imaging apparatus of claim 13, wherein the optical engine includes:
a doublet positioned between the second plano aspheric lens and the camera sensor.

16. The endoscopic imaging apparatus of claim 15, wherein the optical engine includes:
the doublet including a bi-concave aspheric lens cemented to a plano convex lens positioned between the bi-concave aspheric lens and the camera sensor.

17. The endoscopic imaging apparatus of claim 16, wherein a flat surface of the plano convex lens is orientated towards the camera sensor.

18. The endoscopic imaging apparatus of claim 15, wherein the doublet includes a substantially rectangular body extending along the first optical axis.

19. The endoscopic imaging apparatus of claim 11, wherein the pair of imaging modules includes a first imaging module axially aligned with a second imaging module.

20. The endoscopic imaging apparatus of claim 11, wherein the pair of imaging modules includes a first imaging module obliquely aligned with a second imaging module.
